(19) United States
(12) Patent Application Publication
(10) Pub. No.: US 2023/0226048 A1
(43) Pub. Date: Jul. 20, 2023

(54) NEW USE OF RABEXIMOD

(71) Applicant: Cyxone AB, Malmö (SE)

(72) Inventor: Kalev Kask, Mountain View, CA (US)

(21) Appl. No.: 18/000,941

(22) PCT Filed: Jun. 10, 2021
(86) PCT No.: PCT/EP2021/065693

§ 371 Date: Dec. 7, 2022

Related U.S. Application Data
(60) Provisional application No. 63/037,022, filed on Jun. 10, 2020.

(30) Foreign Application Priority Data
Jun. 10, 2020 (EP) 20179239.7
Jun. 10, 2020 (EP) 20179277.7
Jun. 10, 2020 (EP) 20179279.3
Jun. 18, 2020 (EP) 20180706.2

(51) Int. Cl. A61K 31/4985; A61P 29/00

(52) U.S. Cl. CPC A61K 31/4985; A61P 29/00

(57) ABSTRACT

The present invention relates a method for treatment of a pathogenic infection, in particular a pathogenic infection that can lead to an acute respiratory syndrome, such as coronavirus infection, using a composition comprising 9-Chloro-2,3-dimethyl (N,N-dimethylaminoethylamino-2-oxoethyl)-6H-indolo-[2,3-b]quinoxaline (rabeximod) or a pharmaceutically acceptable salt thereof. The present invention also concerns treatment of acute respiratory syndromes, such as ARDS.

### TECHNICAL FIELD

The present invention relates a method for treatment of a pathogenic infection, in particular a pathogenic infection that can lead to an acute respiratory syndrome, such as coronavirus infection, using a composition comprising 9-Chloro-2,3-dimethyl-6-(N, N-dimethylaminoethylamino-2-oxoethyl)-6H-indolo-[2,3-b]quinoxaline (rabeximod) or a pharmaceutically acceptable salt thereof. The present invention also concerns treatment of acute respiratory syndromes, such as ARDS.

### BACKGROUND OF THE INVENTION

Prior to 2002 coronaviruses were not considered to be significant human pathogens. Human coronaviruses known until then, such as HCoV-229E and HCoV-OC43, only cause mild respiratory infections in healthy adults. This perception was shattered in 2002, when severe acute respiratory syndrome coronavirus (SARS-CoV) emerged. This virus rapidly spread to 29 different countries, resulting in 8,273 confirmed cases and 775 (9%) deaths. The implementation of infection control measures brought the epidemic to an end in 2003. In 2012, a novel coronavirus, Middle East respiratory syndrome coronavirus (MERS-CoV), was detected for the first time. To date, 636 laboratory-confirmed cases of MERS-CoV infection have been reported, including 193 deaths, across nine countries. In 2020, a coronavirus known as SARS-Cov-2, first detected in China in December 2019, became a global pandemic. SARS-Cov-2 causes coronavirus disease 2019 (COVID-19), which is (primarily) a respiratory illness.

The clinical features of COVID-19 (as well as of other severe diseases associated with corona virus infections) range from asymptomatic to very severe pneumonia with the potential development of acute respiratory distress syndrome, septic shock, and multi-organ failure resulting in death. Currently, treatment of patients suffering from (severe) respiratory distress syndrome is limited to supportive care, including oxygen support and extracorporeal membrane oxygenation (ECMO), although the benefits of ECMO are still under consideration. ECMO, moreover, is extremely resource-intensive and the recent COVID-19 outbreak is putting healthcare systems throughout the world under extreme strain. The current situation clearly highlights the necessity and value of effective pharmacological treatment of conditions associated with infections by corona viruses and other pathogens causing similar symptoms and conditions.

### SUMMARY OF THE INVENTION

Generally stated, the present invention relates to methods for the therapeutic or prophylactic treatment of subjects in need thereof, in particular subjects suffering from pathogenic infection and/or acute respiratory syndrome that may be associated with such pathogenic infection, using the compound known as rabeximod (9-Chloro-2,3 dimethyl-6-(N,N-dimetylamino-2-oxoethyl)-6H-indolo[2,3-b] quinoxaline), or a pharmaceutically acceptable salt thereof. In an embodiment of the invention, a method is provided for the treatment and/or prevention of said pathogenic infection. In a further embodiment of the invention, a method is provided for the treatment and/or prevention of a symptom or condition associated with said pathogenic infection.

Rabeximod is a pharmacologically optimized follow-on molecule of (2,3-dimethyl-6(2-dimethylaminoethyl)-6H-indolo-[2,3-b]quinoxaline, which was originally developed as an anti-herpes viral drug (Harmenberg et al, 1988, 1991). The compound rabeximod has been described in European patent application publication EP1756111A1 and its US counterpart US 2005/288296. The preparation of rabeximod is specifically described in these patent publications, as compound E.

Based on its strong immunomodulatory and anti-inflammatory properties along with its oral bioavailability, rabeximod was earlier selected as an attractive drug candidate for the treatment of autoimmune diseases such as rheumatoid arthritis (RA). Rabeximod showed anti-arthritic activity in several preclinical models of rheumatoid arthritis. Rabeximod demonstrated statistically significant durable reductions in paw swelling and paw thickness as well as arthritogenic scoring in the murine arthritis model in mice. In a conventional collagen-induced arthritis model in dark agouti rats, daily subcutaneous administration of rabeximod from the day of immunization significantly delayed the onset of clinical arthritis and suppressed arthritis severity.

The use of rabeximod for the treatment of pathogenic infections and associated conditions, in particular pathogenic infections leading to an acute respiratory syndrome, has not been disclosed and/or suggested in the art.

Further objects and advantages of the present invention will appear from the following description, and claims.

### DESCRIPTION OF THE INVENTION

The compound known under the INN ‘rabeximod’ has the IUPAC name 9-Chloro-2,3-dimethyl-6-(N,N-dimethylaminoethylamino-2-oxoethyl)-6H-indolo-[2,3-b]quinoxaline and has the following molecular structure.

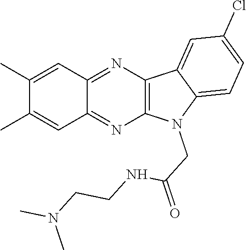 [SMILES: Cc1cc2nc3c4cc(Cl)ccc4n(CC(=O)NCCN(C)C)c3nc2cc1C]

The preparation of Rabeximod is described in EP1756111A1 and US2005/288296. Throughout the present application, the terms “Rabeximod”, “rabeximod” and “9-Chloro-2,3-dimethyl-6-(N, N-dimethylaminoethylamino-2-oxoethyl)-6H-indolo-[2,3-b]quinoxaline” are used interchangeably and mean the compound in any solid form or liquid form unless otherwise indicated or implied under the given circumstances.

Stated generally, the present invention relates to a method for the therapeutic or prophylactic treatment of a subject in need thereof, in particular a subject suffering from a pathogenic infection, more in particular a viral, fungal or bacterial infection, or a subject suffering from an acute respiratory syndrome that may be associated with such a pathogenic infection, wherein said method comprises the administration, to said subject, of a composition comprising 9-Chloro-2,3-dimethyl-6-(N,N-dimethylaminoethylamino-2-oxoethyl)-6H-indolo-[2,3-b]quinoxaline (Rabeximod) or a pharmaceutically acceptable salt thereof. In an embodiment of the invention, said method is a method for the treatment and/or prevention of said pathogenic infection. In an embodiment of the invention, said method is a method for the treatment and/or prevention of a symptom or condition associated with said pathogenic infection. In an embodiment of the invention, said method is a method for the treatment and/or prevention of respiratory syndrome.

Thus, in a first particular aspect, the present invention relates to a method for treatment of a viral or bacterial infection, including nosocomial infections, leading to an acute respiratory syndrome, said method comprising the administration of a composition comprising 9-Chloro-2,3-dimethyl-6-(N, N-dimethylaminoethylamino-2-oxoethyl)-6H-indolo-[2,3-b]quinoxaline (Rabeximod) or a pharmaceutically acceptable salt thereof.

In a second particular aspect, the present invention relates to a method for treatment of an acute respiratory syndrome, optionally associated with pathogenic infection, such as a corona virus infection.

In a third particular aspect, the present invention relates to a method for treatment of a condition leading to inflammatory distress that is characterized by a marked increase in at least one of pro-inflammatory cytokines, accumulation of inflammatory cells, and edema formation, and preferably all of them. These conditions include but are not limited to sepsis of any origin, gastric acid aspiration and pulmonary infections with Anthrax spores. These conditions share similar pathological mechanisms and hence can be alleviated by a blockade of cytokine over activation.

In a fourth particular aspect, the present invention relates to a method of treating a pathogenic infection leading to an acute respiratory syndrome in a mammal, such as a human, comprising administering a therapeutically effective amount of a composition comprising rabeximod or a salt thereof to a mammal in need thereof.

In a fifth aspect the present invention relates to a method of treating an acute respiratory syndrome, optionally associated with a pathogenic infection, such as a corona virus infection in a mammal, such as a human, comprising administering a therapeutically effective amount of a composition comprising rabeximod or a salt thereof to a mammal in need thereof.

In a particular embodiment, the pathogenic infection in the methods defined herein, is a viral infection. Typically, the infection is an infection with a virus selected from the group consisting of influenza viruses (such as influenza A, e.g. H5N1, influenza B and any other annually recurring ones), respiratory syncytial virus, filoviruses, arenaviruses and corona viruses (such as SARS-Cov-1, MERS-Cov or SARS-Cov-2). In a particular embodiment, the infection is an infection with a corona virus, in particular a corona virus selected from the group consisting of SARS-Cov-1, MERS-Cov or SARS-Cov-2. In a particularly preferred embodiment, the infection is SARS-Cov-2 infection.

In a further embodiment, the pathogenic infection in the methods defined herein is a bacterial or fungal infection, such as an infection with Streptococcus pneumoniae or any other bacterial or fungal pathogen that can cause pneumonia.

In a further embodiment the viral or bacterial infection in the methods defined herein is an infection with a nosocomial pathogen, such as a nosocomial species of bacteria or fungus.

In an embodiment of the invention, a method as defined herein is provided, for the treatment and/or prevention of said pathogenic infection, more in particular for the treatment of said pathogenic infection.

In an embodiment of the invention, a method as defined herein is provided, for the treatment and/or prevention of a symptom or condition associated with said pathogenic infection. In a still further embodiment, the symptom or condition associated with the pathogenic infection is acute respiratory syndrome, in particular Acute Lung Injury (ALI), more in particular Acute Respiratory Distress Syndrome (ARDS), which is the most severe form of ALI. In a still further embodiment the symptom or condition associated with the pathogenic infection is Corona virus disease 2019 (COVID-19). In a still further embodiment the symptom or condition associated with the pathogenic infection is pneumonia.

Preferably, the method of the present invention is a method for treatment of a corona viral infection, such as Covid-19, in a human subject. In another preferred embodiment, the method of the present invention is a method for treatment of an acute respiratory syndrome, such as ARDS, e.g. ARDS caused by a viral infection, such as Covid-19.

In the methods as defined herein, the subject is typically a mammal. In a preferred embodiment, the subject to be treated is a human subject. In a particularly preferred embodiment of the invention, the subject to be treated is a human subject that is at increased risk of a severe course of disease following the pathogenic infection, such as a subject having an age of above 25 years, above 40 years, above 50 years, above 55 years, above 60 years or above 65 years; a subject suffering from a certain pathology correlating with a severe course of disease, such as a pathology selected from cardiovascular disease, diabetes, obesity, chronic obstructive pulmonary disease (COPD) and/or high blood pressure; a subject that is at increased risk of a severe course of disease due to genetic predisposition; a subject that is at increased risk of a severe course of disease due to certain life-style habits, such as smoking and/or being overweight as a consequence of unhealthy diet; or a subject having a biomarker profile that is indicative of increased risk of a severe course of disease. In certain embodiments, the present methods comprise the step of identifying subjects that are at increased risk of suffering a severe course of disease following the pathogenic infection. In certain embodiments, the present methods comprise the step of diagnosing or establishing whether a subject is at increased risk of suffering a severe course of disease following the pathogenic infection.

In an embodiment, the administration of rabeximod in the methods as defined herein result in the inhibition of the release of the cytokines IL-6, TNFα, GM-CSF, IL-1 ß, and chemokines IL-8/CXCL8, MIP-1α/CCL3, MIP-11ß/CCL4, MCP-1/CCL2 from LPS-induced macrophages.

Accordingly, a further aspect the present invention relates to a method for treatment and/or prevention of a condition involving inflammatory distress characterized by a marked increase in at least one of pro-inflammatory cytokines, accumulation of inflammatory cells, and edema formation, said method comprising administering a composition comprising 9-Chloro-2,3-dimethyl-6-(N,N-dimethylaminoethylamino-2-oxoethyl)-6H-indolo-[2,3-b]quinoxaline (Rabeximod) or a pharmaceutically acceptable salt thereof. These conditions include but are not limited to sepsis of any origin, gastric acid aspiration and pulmonary infections with Anthrax spores. These conditions share similar pathological mechanisms and hence can be alleviated by a blockade of cytokine over activation.

In an embodiment, the methods as defined herein comprise the administration of a composition comprising rabeximod in the form of the free base. Preferably, the rabeximod is a crystalline free base having a melting point of 259-261° C. In another embodiment the methods comprise the administration of rabeximod in the form of a pharmaceutically acceptable salt.

In an embodiment, the methods as defined herein comprise the daily administration of the composition, typically via the enteral route of administration. Preferably, the method comprises the administration of rabeximod at a dosage of 6-600 mg, such as form 6.25 mg to 12.5 mg, 12.5 mg to 15 mg, 15 mg to 25 mg, 25 mg to 37.5 mg, 37.5 mg to 50 mg, 50 mg to 100 mg, 100 mg to 200 mg, 200 mg to 400 mg, or the administration of a pharmaceutically acceptable salt of rabeximod at the equivalent or equipotent dosage. As used herein, the term “equipotent” means equally potent or equally capable of producing a pharmacologic effect of certain intensity. For example, if the composition comprises a salt of rabeximod the amount of said salt to be administered typically needs to be adjusted to take account of the molecular weight difference between the free base and salt form. It is also common in the art to refer to amounts of a given compound “equivalent” to a specified amount of a reference compound. For instance, in expressing dose amounts in the label and/or product information of authorized medicinal products comprising a salt form of an active compound that can also be used in free base form, it is customary practice to specify the dose of the free base that the dose of the salt is equivalent to. In this context, the term ‘equipotent’ is deemed synonymous to the term ‘equivalent’.

Any of the mentioned dosages can be administered one or more times daily, however it is preferred to administer the dosage once daily. Thus, any one of the dosages from 6-600 mg rabeximod may be administered once daily, such as about 15 mg rabeximod once daily.

In order to optimize treatment of a human suffering from a pathogenic infection, such as a viral or bacterial infection, leading to an acute respiratory syndrome, e.g. a corona viral infection, such as Covid-19, or a an acute respiratory syndrome, optionally associated with a viral or bacterial infection, the method may comprise additional treatment with another pharmacologically active agent, for example other substances displaying antiviral properties including but not limited to nucleotide and nucleoside analogs (merimepodib, remdesivir, EIDD-2801) and other inhibitors of viral life cycle (umifenovir, camostat mesilate or nafamostat mesilate, zotatifin, plitidepsin, baricitinib, ivermectin, oseltamivir, zanamivir). Any one of the specified other medicaments are subject to individual embodiments in combination with any one of first to fifth aspects as described above and any of the embodiments hereof. In case of a corona viral infection, such as Covid-19, or a respiratory syndrome, such as ARDS, Remdezivir or Merimepodib may be used.

The composition comprising rabeximod or a pharmaceutically acceptable salt thereof may be administered before, simultaneously or after administration of the second medicament. Typically, the composition comprising rabeximod or a pharmaceutically acceptable salt thereof is administered to a human.

In further aspects, the present invention relates to a composition comprising 9-Chloro-2,3-dimethyl-6-(N, N-dimethylaminoethylamino-2-oxoethyl)-6H-indolo-[2, 3-b]quinoxaline (Rabeximod) or a pharmaceutically acceptable salt thereof for use in any of the methods as defined herein.

In yet further aspects, the present invention relates to the use of a composition comprising 9-Chloro-2,3-dimethyl-6-(N, N-dimethylaminoethylamino-2-oxoethyl)-6H-indolo-[2,3-b]quinoxaline (Rabeximod) or a pharmaceutically acceptable salt thereof for the manufacture of a medicament for use in any of the methods as defined herein.

As stated herein before, the compositions suitable and/or intended for use in any of the afore defined methods may comprise rabeximod in the form of the free base. Preferably, the rabeximod is a crystalline free base having a melting point of 259-261° C. In another embodiment the compositions comprise rabeximod in the form of a pharmaceutically acceptable salt.

Preferably, the composition is a composition suitable for enteral administration, such as an oral solid composition, e.g. a tablet or capsule.

In particular embodiments of the invention, the compositions suitable and/or intended for use in any of the afore defined methods of treatment comprise rabeximod in an amount of 6-600 mg, such as form 6.25 mg to 12.5 mg, 12.5 mg to 15 mg, 15 mg to 25 mg, 25 mg to 37.5 mg, 37.5 mg to 50 mg, 50 mg to 100 mg, 100 mg to 200 mg, 200 mg to 400 mg, or a pharmaceutically acceptable salt of rabeximod in the equivalent or equipotent amount.

In an embodiment the composition comprises a pharmaceutically acceptable additive. As used herein “pharmaceutically acceptable additive” is intended without limitation to include carriers, excipients, diluents, adjuvant, colorings, aroma, preservatives etc. that the skilled person would consider using when formulating rabeximod in order to make a pharmaceutical composition.

The adjuvants, diluents, excipients and/or carriers that may be used in the composition of the invention must be pharmaceutically acceptable in the sense of being compatible with rabeximod and the other ingredients of the pharmaceutical composition, and not deleterious to the recipient thereof. It is preferred that the compositions shall not contain any material that may cause an adverse reaction, such as an allergic reaction. The adjuvants, diluents, excipients and carriers that may be used in the pharmaceutical composition of the invention are well known to a person within the art. The pharmaceutically acceptable additive is typically selected form one or more of a filler, glidant and lubricant, as long as the additive do not affect stability of the rabeximod. Typical filler is Microcrystalline cellulose (Avicel PH-102) or (Avicel PH-200). Typical glidant is Silica colloidal anhydrous (Aerosil 200). Typical lubri-cant is Magnesium stearate.

In an embodiment the composition comprises a further pharmacologically active agent, for example other agents displaying antiviral properties including but not limited to nucleotide and nucleoside analogs (merimepodib, remdesivir, EIDD-2801) and other inhibitors of viral life cycle (umifenovir, camostat mesilate or nafamostat mesilate, zotatifin, plitidepsin, baricitinib, ivermectin, oseltamivir, zanamivir). In case of a composition intended for treatment of corona virus infection and/or a symptom or condition associated there with, such as Covid-19, or a respiratory syndrome, such as ARDS, the further pharmacologically active agent may be Remdezivir and/or Merimepodib.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or dis-order and includes the administration of the active compounds to prevent the onset of the symptoms or complications. The treatment may either be performed in an acute or in a chronic way. The patient to be treated is preferably a mammal; in particular, a human being, but it may also include animals, such as dogs, cats, cows, sheep and pigs.

The term “and/or” as used herein is intended to mean both alternatives as well as each of the alternatives individually. For instance, the expression “xxx and/or yyy” means “xxx and yyy”; “xxx”; or “yyy”, all three alternatives are subject to individual embodiments.

The above embodiments should be seen as referring to any one of the aspects (such as composition comprising Rabeximod’ or ‘method of treating a corona viral infection’) described herein as well as any one of the embodiments described herein unless it is specified that an embodiment relates to a certain aspect or aspects of the present invention.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way.

Any combination of the above-described elements in all possible variations thereof is encompassed by the invention unless otherwise indicated herein or otherwise clearly contradicted by context.

The terms “a” and “an” and “the” and similar referents as used in the context of de-scribing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Unless otherwise stated, all exact values provided herein are representative of corresponding approximate values (e.g., all exact exemplary values provided with respect to a particular factor or measurement can be considered to also pro-vide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition de-scribed herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

The present invention is further illustrated by the following examples that, however, are not to be construed as limiting the scope of protection. The features disclosed in the foregoing description and in the following examples may, both separately and in any combination thereof, be material for realizing the invention in diverse forms thereof.

### EXPERIMENTAL

### Experiment 1—Rabeximod Decreases IL-6 Values in Humans

In a phase IIa clinical trial, a total of 225 patients diagnosed with active rheumatoid arthritis were randomized to receive one of the following treatments:
6.25 mg of Rabeximod (daily)
15 mg of Rabeximod (daily)
37.5 mg of Rabeximod (daily)
Placebo

Study drug was provided in the form of a capsule comprising rabeximod in the form of a (micronized) powder. Over 200 patients completed the study.

The primary aim of the study was to evaluate the safety and efficacy of

Rabeximod treatment in RA patients. The study also involved Pharmacokinetic and Pharmacodynamic assessments in a subgroup (PK/PD Population) of 36 patients. This included assessment of changes in hsCRP, TNF-α, IL-6, COMP and anti-CCP.

Individual patient IL-6 values varied notably over the course of the study, with Values ranging from 52.0 IU/L to 19840 IU/L. This degree of variation was seen across treatment groups. Median IL-6 (IU/L) values were decreased from baseline at the majority of time points across all treatment groups over the course of the study. At Week 12, changes in IL-6 median values were −185.0, −1679.0, and 136.0 IU/L in the Rabeximod 6.25, 15 and 37.5 mg groups, respectively and −731.5 IU/L in the placebo group. At Week 16 a decrease in median IL-6 values from baseline was noted across all treatment groups. Changes in IL-6 median values were −804, −1879, and −1124 IU/L in the Rabeximod 6.25, 15 and 37.5 mg groups, respectively, and −1756 IU/L in the placebo group.

### Experiment 2—Rabeximod Effect on Cytokine/Chemokine Release In Vitro

The effect of Rabeximod on the release of GM-CSF, IL-113, IL-6, IL-8, TNF-α, MCP-1, MIP-1a, MIP-1β and RANTES from human peripheral blood mononuclear cells (PBMC) in vitro was evaluated. Rabeximod was tested at two concentrations (1.25 μg/ml and 12.5 μg/ml) on PBMC's isolated from 7 donors. Human PBMCs were isolated from whole blood using a Ficoll gradient. Cells (26106/ml) were then plated in a volume of 0.5 ml (0.5 ml of vehicle control (1% methanol in RPMI 1640 media) or rabeximod resuspended in methanol and diluted to a concentration of 2.5 or 25 mg/ml media was added). After incubation (1 h 37° C.) with LPS (0.1 mg/ml), cells were further incubated for 18-20 h before supernatant was removed for analysis of cytokine production using cytokine multiplex assays (Biosource, Nivelles, Belgium), accord-ing to the manufacturer's instructions. Data were collected using a Luminex 100 (Luminex Corporation, Austin, Tex., USA). All samples were analyzed in duplicates and a mean value for each sample was calculated.

Rabeximod was found to decrease the LPS-stimulated production of pro-inflammatory cytokines and chemokines. IL-6, TNFα, MIP-1α and MIP-1β were significantly reduced by low levels of Rabeximod (1.25 μg/ml), while all but RANTES were reduced by high levels 12.5 μg/ml) of Rabeximod.

### Experiment 3—a Study on the Effect of Rabeximod on Toll-Like Receptor (TLR) 2 and 4 Activation Mediated Activation of Macrophages In Vitro/Ex Vivo

The aim was to investigate whether, after stimulation, Rabeximod has the ability to decrease production of pro-inflammatory cytokines by macrophages isolated from the peritoneal cavity after thioglycolate recruitment.

B10.Q mice with/without expression of functional TLR4 matched according to sex and age were used as donors of peritoneal macrophages. Mice were injected on day −5 with sterilized 3% Thioglycolate intraperitoneally. On day 0 the peritoneal cavity was washed with ice cold PBS to collect recruited cells. The cells were counted and added to plates at a final concentration of 200 000 cells/well in medium. Rabeximod diluted in a minimal amount DMSO and then DMEM to final concentration (12.5 ug/ml) was added simultaneously (h 0) or at time points+0.5 h, +1 h or +3 hours after LPS or TLR2 stimuli (Lipomannan). Cells were incubated for 24 hours before the whole plate was frozen. TNF-α ELISA was performed after one week using antibodies from e-biosciences and detected by europium.

In Vitro Activation of Macrophages

Mice were injected on day 0 with 5% thioglycolate (1 ml) intraperitoneally. On day 3 after injection, recruited cells were washed out with ice-cold PBS and plated at a concentration of 2×10⁶ cells/well in cell culture medium containing penicillin/streptomycin, heat-inactivated fetal calf serum (5%), 1% 4-(2-hydroxyethyl)-1-piperazineethanesulfonic acid (HEPES) (1 M) and 0.1% β-mercaptoethanol (50 mM).

Results of this investigation show that Rabeximod decreases TNF-α production from TLR2 stimulated macrophages with stronger effect if administered early after stimulation.

### Experiment 4—a Study on Mixed Lymphocytes Reaction (MLR) of 12 Human Donors in the Presence of Rabeximod

This study examined the effect of Rabeximod on mixed lymphocytes reaction (MLR) of human donors. MLR is used for the diagnosis of immunodeficiency disease.

Peripheral blood mononuclear cells (PBMCs) were prepared and modified for use as stimulator cells through γ-irradiation, or for use as responder cells. Rabeximod was then added to a responder and incubator cell mix at various concentrations (of 10⁻⁵, 10⁻⁶, 10⁻⁷ and 10⁻⁸ M) before the cells were incubated. In the test, responder cells should proliferate more in the presence than in the absence of stimulator cells.

Rabeximod was diluted in a minimal amount of dimethylsulfoxide (DMSO) and Dulbecco's modified Eagle medium (DMEM) to a final concentration of 12.5 μg/ml and added simultaneously (0 h) or at time points+0.5 h, +1 h or +3 h after LPS (0.1 μg/ml in medium) or TLR2 stimuli (LM, 1 μg/ml in medium). Cells were incubated for 24 h before the whole plate was frozen. TNFα production was determined using a ready-SET-go! ELISA kit from E-bioscience (E-bioscience, San Diego, Calif., USA).

Normal PBMCs were obtained from healthy volunteers at the blood bank (Shiba medical center, Tel-Hashomer). Blood donations tested negative to HBSAg, HIV, HCV, ALT, HTLV and TPHA. Twelve donors were obtained in 3 sessions following review by the Committee for Ethical Conduct in the Care and Use of Laboratory Animals of the Hebrew University, Jerusalem, the Institutional Animal Care and Use Committee (IACUC) responsible for approving Harlan Biotech (Israel) animal usage application in compliance with its respective registration under National Institues of Health (NIH) accreditation no. OPR-A01-5011. Peripheral blood mononuclear cells (PBMCs) were freshly separated from heparinized full blood by Ficoll-Hypaque (IsoPrep, Robbins Scientific) density gradient centrifugation. After two washes with PBS, cells were diluted in PBMCs medium and counted. Since MLR was conducted in two-ways format, each donor was utilized as stimulator and as responder.

In each session, 0.87*10⁶/ml PBMCs cells from each donor were mixed and then γ-irradiated with 4000 rad (about 6.25 minutes). Cells were diluted in PBMCs medium to give the highest concentration). Further dilutions in ×2-fold were done in PBMCs medium. 50*10⁶ were taken from each donor to obtain a mixture of stimulators in each experiment.

The results of the study show that Rabeximod displayed a concentration-dependent inhibition of MLR activity with a clear dose response. At the highest concentration (10⁻⁵ M) strong inhibitory effect of MLR activity was observed in all responder:stimulator tested ratios, while at a lower concentration (10⁻⁸ M) the inhibition decreased to a lesser extent. At all tested ratios, the two highest concentration of Rabeximod were highly inhibitory. Lower doses were less effective. From these results it can be concluded that Rabeximod decreases the immune response when blood from different donors is mixed.

### Experiment 5—a Study on the Effect of Rabeximod on Toll-Like Receptor (TLR) 4 and 2 Activation In Vivo

QB mice (10-20 weeks) were generated from a (BALB/cxB10.Q) F1 cross. C57BL/10ScNJ mice (Tlr4^{lps-del}), hereafter called Tlr4^{del}, were ordered from The Jackson Laboratory (The Jackson Laboratory, Bar Harbor, Me., USA). These mice have deletion of the Tlr4 gene that results in absence of mRNA and protein and is thus defective in response to LPS stimulation. These mice were crossed together with C57BL/10.Q mice from Medical Inflammation Research, Lund university, Lund, Sweden in order to generate mice expressing major histocompatibility complex (MHC) Aq. Mice were kept and bred in a climate-controlled environment with a 12 h light/dark cycle, fed standard rodent chow and water ad libitum in the animal facility of Medical Inflammation Research, Lund University, Lund, Sweden.

Litters were genotyped for MHC using microsatellite marker D17mit230. Genotyping of Tlr4 was performed according to an earlier described method. Briefly, DNA was prepared from biopsies and primers flanking the 70 kb deletion or amplifying the wild-type sequence absent in the TLR4 deleted mice were used.

Deletion primer pair: sense 5′-GCAAGTTTCTATATGCATTCT-3′, antisense 5′-CCTCCATTTCCAATAGGTAG-3′, generating a 140 bp amplicon; 80K primer pair: sense 5′-ATATGCATGATCAACACCACA-3′, antisense 5′-TTTCCATTGCTGCCCTATAG-3′, generating a 390 bp amplicon.

The products from the PCRs were run on a 1.5% polyacrylamide gel to determine length of fragments.

The mice were matched according to age and sex, and littermates were used in all experiments. All experiments were approved according to Malmö/Lund ethical committee license M107-07.

Immunization Protocol

Male mice, older than 8 weeks, were injected intravenously on day 0 with a cocktail of four monoclonal antibodies (4 mg in a total volume of 310 μl/mouse) containing M2139, CIIC1, CIIC2 and UL1 antibodies binding to J1, C1^{I}, D3 and U1 epitopes of CII in order to induce CAIA. Day 5 mice were injected with LPS from Escherichia coli (Sigma-Aldrich, St Louis, Mis-souri, USA), deoxycytoylate-phosphate-deoxyguanylate (CpG) DNA (sequence 1668, MWG; Eurofins MWG, Erdersberg, Germany) or lipomannan (LM) from Mycobacterium segma-tis (InvivoGen, San Diego, Calif., USA) intraperitoneally to boost disease (35-50 μg/mouse in 1 ml phosphate-buffered saline (PBS)).

Scoring Protocol

Mice were blindly scored daily to every second day until the end of the experiment. The scoring protocol used is described in detail in Holmdahl et al. In this protocol each paw can get a maximum of 15 scores and each mouse can get a maximum of 60 scores.

Treatment Protocol

Rabeximod was dissolved in corn oil (Sigma Aldrich) to a concentration of 30 mg/ml and ultra-sonicated in a water bath (68° C.) for 2×30 min. Rabeximod in corn oil was injected subcutaneously in the back (40 mg/kg) with the first injection on day 5 after antibody injection (just after TLR stimulation) and continued for six injections with injections every second day until day 15.

Results

Rabeximod reduces arthritis severity in both wild type and TLR4 deficient mice and inhibits activation of macrophages in a downstream mechanism in animal models of arthritis, likely by affecting TLR2 signalling pathway.

### Experiment 6—a Study on the Effects Rabeximod on Proliferative Responses by Lymph Node Cells Derived from Immunized Rats

The objective of this study was to investigate the suppressive effect of Rabeximod on lymphocytes activation.

Cells were resuspended in PBMCs medium at approximately 2*10⁶ cells/ml and incubated in flasks for 2 hours at 5% CO₂, 37° C. Adherent cells were dis-carded while non-adherent cells in the supernatant were centrifuged at 700 g for 10 minutes. To enrich for T lymphocytes, cells were resuspended in 5% PBMCs medium and loaded on nylon wool columns (Uni-Sorb, Novamed) at 8*10⁷ cells/ml, according to manufacturer instructions.

Cells were seeded in 96-well round-bottom plates at the varying ratios of stimulator vs. responder cells.

³[H]-Thymidine was added to the cell culture at 18 hrs before the end of the cultivation period (6 days). Cells were harvested and transferred to filters (unifilters GF/C) using the Packard filtermate Cell harvester. Following addition of scintillation fluid (MicroScint20™) to the filters, they were counted in Packard microplate scintillation β-counter. Results are expressed as Counting Per Minutes (CPM).

Eleven Responders and stimulators cells were incubated together in the presence of rabeximod. the effect of rabeximod on MLR activity was similar in all the responder:stimulators tested ratios. Rabeximod displayed concentration-dependent inhibition at a dose response manner. At the highest concentration (10⁻⁵ M) strong inhibitory effect was observed, while at the lowest rabeximod concentration (10⁻⁸ M) the inhibition decreased to a lesser extent. At all tested ratios of Rob-803 the two highest concentrations were highly inhibitory, and the two lowest ones were less effective.

In order to study suppressive effects of rabeximod on T cell activity the proliferation assays were set up as follows:

draining lymph nodes collected day 9 p.i. from DA rats immunized with rat collagen II (RCII) in Freund's Incomplete Adjuvant (FIA) were used to prepare individual single cell suspensions. Lymph nodes were grinded through wire mesh and cells washed 3 times in incomplete DMEM cell culture medium. Cells were counted by trypan blue exclusion and cell suspensions of 2×10⁶ cells/ml in complete DMEM prepared. 4×10⁵ cells per well were seeded in flat-bottomed 96-well microtiter plates. Triplicates of each individual cell suspension were incubated with 1.5 μg/ml ConA. ConA was diluted in phosphate buffered saline, D-PBS. Spontaneous proliferation was determined by measuring proliferation in cell cultures without addition of ConA. As a positive proliferation control cells were stimulated with ConA together with 10 μl of the rabeximod vehicle, 0.01 M acetic acid. Doses of rabeximod were added in 10 μl vehicle so that the final concentrations in cell cultures ranged from 50-800 ng/ml. Cell cultures were incubated at 37° C.+5% CO₂ for 48 h and 1 μCi of [³H]-Thymidine per well was added to the cultures for the last 12 h. Cellular incorporation of [³H]-thymidine was measured using a 1450 Microbeta Wallac Trilux Liquid Scintillation Luminescence Counter after harvesting the cells onto nitrocellulose filters.

Individual Inhibition Indices (1.1.) were calculated by dividing the average counts per minute (cpm) value of each triplicate stimulated with ConA and rabeximod with the average cpm value of the triplicate stimulated with ConA only. The inhibition of proliferation induced by rabeximod addition was expressed as % of the positive control (ConA only), i.e. by multiplying the inhibition indices with 100.

The in vitro inhibitory effect of rabeximod on T cell proliferation was investigated for concentrations of rabeximod ranging between 50 and 800 ng/ml. We observed that 300 ng/ml of rabeximod inhibited T cell proliferation to 50% or more. When the concentration of rabeximod was increased to 500 ng/ml, the inhibition increased to 80% or more. Results from this study showed that Rabeximod has an inhibitory effect on T cell proliferation in vitro. The T cells were derived from rats immunized with rat collagen II and stimulated in vitro with a T cell mitogen, Con A. Approximately 50% inhibition of the proliferation was observed at the concentration of 300 ng/ml Rabeximod. An increase of the Rabeximod dose to 500 ng/ml increased inhibition to approximately 80%. When determining the effective dose, one should consider that earlier reports have defined that approximately 99% of Rabeximod in plasma is bound to protein and might, thus, not be effective.

### REFERENCES

Beigel J H, Farrar J, Han A M, et al. Avian influenza A (H5N1) infection in humans. N Engl J Med. 2005; 353(13):1374-1385.
Billack B. Macrophage activation: role of toll-like receptors, nitric oxide, and nuclear factor kappa B. Am J Pharm Educ. 2006; 70(5):102.
Cheung C Y, Poon L L, Lau A S, et al. Induction of proinflammatory cytokines in human macrophages by influenza A (H5N1) viruses: a mechanism for the unusual severity of human disease?. Lancet. 2002; 360(9348):1831-1837.
Corman V M, Landt O, Kaiser M, et al. Detection of 2019 novel coronavirus (2019-nCoV) by real-time RT-PCR. Euro Surveill. 2020; 25(3):2000045.
Guarner J, Jernigan J A, Shieh W J, et al. Pathology and pathogenesis of bioterrorism-related inhalational anthrax. Am J Pathol. 2003; 163(2):701-709.
Haeberle H A, Takizawa R, Casola A, et al. Respiratory syncytial virus-induced activation of nuclear factor-kappaB in the lung involves alveolar macrophages and toll-like receptor 4-dependent pathways. J Infect Dis. 2002; 186(9):1199-1206.
Harbecke O, Dahlgren C, Bergman J, Möller L. The synthetic non-toxic drug 2,3-dimethyl-6(2-dimethylaminoethyl)-6H-indolo-(2,3-b)quinoxaline inhibits neutrophil production of reactive oxygen species. J Leukoc Biol. 1999; 65(6):771-777.
Harmenberg J, Wahren B, Bergman J, Akerfeldt S, Lundblad L. Antiherpesvirus activity and mechanism of action of indolo-(2,3-b)quinoxaline and analogs. Antimicrob Agents Chemother. 1988; 32(11):1720-1724.
Harmenberg J, Akesson-Johansson A, Gräslund A, et al. The mechanism of action of the anti-herpes virus compound 2,3-dimethyl-6(2-dimethylaminoethyl)-6H-indolo-(2,3-b)quinoxaline. Antiviral Res. 1991; 15(3):193-204.
Hemmi H, Takeuchi O, Kawai T, et al. A Toll-like receptor recognizes bacterial DNA. Nature. 2000; 408(6813):740-745.
Lew T W, Kwek T K, Tai D, et al. Acute respiratory distress syndrome in critically ill patients with severe acute respiratory syndrome. JAMA. 2003; 290(3):374-380.
McGonagle D, Sharif K, O'Regan A, Bridgewood C. The Role of Cytokines including Interleukin-6 in COVID-19 induced Pneumonia and Macrophage Activation Syndrome-Like Disease. Autoimmun Rev. 2020; 19(6):102537.
Peiris J S, Yu W C, Leung C W, et al. Re-emergence of fatal human influenza A subtype H5N1 disease. Lancet. 2004; 363(9409):617-619.
Tumpey T M, Basler C F, Aguilar P V, et al. Characterization of the reconstructed 1918 Spanish influenza pandemic virus. Science. 2005; 310(5745):77-80. van Riel D, Munster V J, de Wit E, et al. H5N1 Virus Attachment to Lower Respiratory Tract. Science. 2006; 312(5772):399.
Westman E, Thi Ngoc D D, Klareskog L, Harris H E. Suppressive effects of a quinoxaline-analogue (Rob 803) on pathogenic immune mechanisms in collagen-induced arthritis. Clin Exp Immunol. 2008; 152(1):192-199.

1. A composition comprising 9-Chloro-2,3-dimethyl-6-(N,N-dimethylaminoethylamino-2-oxoethyl)-6H-indolo-[2,3-b]quinoxaline (Rabeximod) or a pharmaceutically acceptable salt thereof for use in a method for treatment of a viral or bacterial infection, including nosocomial infections, leading to an acute respiratory syndrome.

2. The composition for use of claim 1 wherein rabeximod is the free base.

3. The composition for use of any one of claims 1-2 wherein the composition is administered daily, such as orally.

4. The composition for use of claim 3 wherein the daily dosage is from 6-600 mg rab-eximod or a pharmaceutically acceptable salt thereof.

5. The composition for use of claim 4 wherein the daily dosage is administered once daily.

6. The composition for use of any one of claims 1-5 wherein the viral or bacterial infection is in a human subject.

7. The composition for use of any one of claims 1-6 wherein the viral or bacterial infection is a viral infection, such as a corona viral infection.

8. The composition for use of claim 7 wherein the viral infection is influenza, pneumonia, SARS, MERS or Covid-19.

9. The composition for use of any one of claims 1-6 wherein the viral or bacterial infection is a bacterial infection.

10. The composition for use of claim 9 wherein the bacterial infection is pneumonia.

11. The composition for use of any one of claims 1-10 wherein the acute respiratory syndrome is ALI or ARDS.

12. A composition comprising 9-Chloro-2,3-dimethyl-6-(N,N-dimethylaminoethylamino-2-oxoethyl)-6H-indolo-[2,3-b]quinoxaline (Rabeximod) or a pharmaceutically acceptable salt thereof for use in a method for treatment of an acute respiratory syndrome, such as ALI or ARDS, optionally associated with a viral or bacterial infection, such as a corona viral infection.

13. A method for treatment of a viral or bacterial infection, including nosocomial infections, leading to an acute respiratory syndrome, wherein said method comprises administering a composition comprising 9-Chloro-2,3-dimethyl-6-(N,N-dimethylaminoethylamino-2-oxoethyl)-6H-indolo-[2,3-b]quinoxaline (Rabeximod) or a pharmaceutically acceptable salt thereof.

14. A method for treatment of an acute respiratory syndrome, such as ALI or ARDS, optionally associated with a viral or bacterial infection, such as a corona viral infection, wherein said method comprises administering a composition comprising 9-Chloro-2,3-dimethyl-6-(N,N-dimethylaminoethylamino-2-oxoethyl)-6H-indolo-[2,3-b]quinoxaline (Rabeximod) or a pharmaceutically acceptable salt thereof.

15. A method of treating a subject suffering from viral or bacterial infection, including nosocomial infection, wherein said method comprises administering a composition comprising 9-Chloro-2,3-dimethyl-6-(N, N-dimethylaminoethylamino-2-oxoethyl)-6H-indolo-[2,3-b]quinoxaline (Rabeximod) or a pharmaceutically acceptable salt thereof.
